(19) United States
(12) Patent Application Publication
(10) Pub. No.: US 2008/0207679 A1
(43) Pub. Date: Aug. 28, 2008

(54) Glutathione peroxidase mimetics for the treatment of dermatoses

(76) Inventor: Noah Berkowitz, New Rochelle, NY (US)

Correspondence Address: Pearl Cohen Zedek Latzer, LLP, 1500 Broadway, 12th Floor, New York, NY, 10036, US

(21) Appl. No.: 12/071,272

(22) Filed: Feb. 19, 2008

Related U.S. Application Data
(60) Provisional application No. 60/901,678, filed on Feb. 16, 2007.
(60) Provisional application No. 61/006,444, filed on Jan. 14, 2008.

(51) Int. Cl. A61K 31/4353; A61P 17/02

(52) U.S. Cl. 514/300

(57) ABSTRACT

This invention relates to compositions and methods for the treatment of dermatological conditions with a glutathione peroxidase mimetic.

### CROSS-REFERENCE TO RELATED APPLICATIONS

This application claims priority under 35 U.S.C. §119(e) to U.S. provisional patent applications Ser. Nos. 60/901,678, filed Feb. 16, 2007, and 61/006,444, filed Jan. 14, 2008. Both applications are incorporated herein by reference in their entireties.

### FIELD OF INVENTION

This invention is directed to compositions and methods for the treatment of dermatoses with glutathione peroxidase and its mimetics.

### BACKGROUND OF THE INVENTION

Many skin disorders are treated with a single course of therapy on the premise that the etiology and presented symptoms are the result of a single cause. Unfortunately, many diseases, especially skin diseases, are complicated in that the symptoms may be the result of changes in internal, external, or a combination of both environments. Consequently, conventional single agent therapies do not yield the desired clinical results demonstrated, for example, as cosmetic improvement (appearance), elimination of pathogenic organisms, reduction of swelling, etc. Skin disorders where two or more conditions have been identified include acne and rosacea.

Antibacterial compositions for dermatological treatment such as those used for acne, must remain stable for long periods of time without losing potency nor form insoluble substances or complexes because of the combining of active ingredients, as well as not being irritating to the skin. Likewise, side effects from the use of topical steroids have become more prevalent since the introduction of the higher potency topical steroids. Using these products on thin or denuded skin, on the elderly or pediatric population, or under occlusion increases the incidence of side effects. Striae and atrophy, which are the most commonly observed side effects, occur with prolonged use and are more likely to occur in areas of sweating, occlusion, or high penetration such as the axilla or groin. In general, atrophy does not occur until the agent has been used for 3 to 4 weeks and is usually reversible. Striae, which develop when the weakened skin is stretched, are not reversible. Prolonged treatment can also result in “steroid acne,” which is characterized by crops of dense, inflamed pustules in the same developmental stage. These lesions occur on the face, chest and back. Perioral and periocular dermatitis have been associated with the use of topical steroids and usually improve with the cessation of the steroid. Other topical treatments such as coal tar, and keratolytics can be cumbersome, and have limited efficacy.

These therapies have some disadvantages; in many people, some antihistaminics cause languor and drowsiness. Permanent use of glucocorticoids (for example cortisone) is usually unjustifiable for medical reasons owing to many unpleasant side effects. The same also applies to most so-called NSAIDs (non-steroidal anti-inflammatory drugs), which exhibit several undesirable side effects, such as gastric bleeding, upset stomach and cardiac risk. Due to the debilitating nature of dermatoses there continues to exist a need for effective treatments.

### SUMMARY OF THE INVENTION

In one embodiment, the invention provides a composition for treating a dermatological condition in a subject, comprising: a glutathione peroxidase mimetic or an isomer, metabolite, and/or salt thereof, and pharmaceutically acceptable carrier or diluent.

In another embodiment, the invention provides a method of treating a dermatological condition in a subject, comprising the step of contacting said subject with a therapeutically effective amount of a composition comprising a glutathione peroxidase mimetic or its isomer, metabolite, and/or salt thereof and pharmaceutically acceptable carrier or diluent.

In one embodiment, the invention provides a method for inhibiting or suppressing a dermatological condition in a subject, comprising the step of contacting the subject with a glutathione peroxidase mimetic or its isomer, metabolite, and/or salt thereof and pharmaceutically acceptable carrier or diluent.

In another embodiment, the invention provides a method for reducing the incidence of a dermatological condition in a subject, comprising the step of contacting the subject with a glutathione peroxidase mimetic or its isomer, metabolite, and/or salt thereof and pharmaceutically acceptable carrier or diluent.

In one embodiment, the invention provides a method of treating a dermatological condition in a subject, comprising the step of contacting said subject with the compound represented by formula I and a pharmaceutically acceptable carrier or diluent.

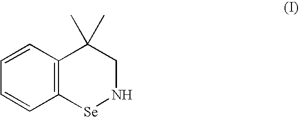

In one embodiment, the glutathione peroxidase mimetic or its isomer, metabolite, and/or salt thereof is represented by the compound of formula (II):

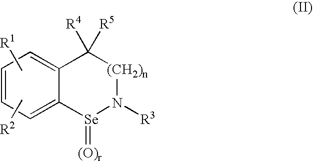

wherein R¹ and R² are independently hydrogen; lower alkyl; OR⁶; —(CH₂)ₘNR⁶R⁷; —(CH₂)_{q}NH₂; —(CH₂)ₘNHSO₂(CH₂)₂NH₂; —NO₂; —CN; —SO₃H; —N⁺(R⁵)₂O⁻; F; Cl; Br; I; —(CH₂)ₘR⁸; —(CH₂)ₘCOR⁸; —S(O)NR⁶R⁷; —SO₂NR⁶R⁷; —CO(CH₂)ₚCOR⁸; R⁹;
R³=hydrogen; lower alkyl; aralkyl; substituted aralkyl; —(CH₂)ₘCOR⁸; —(CH₂)_{q}R⁸; —CO(CH₂)ₚCOR⁸; —(CH₂)ₘ SO₂R⁸; —(CH₂)ₘS(O)R⁸;
R⁴=lower alkyl; aralkyl; substituted aralkyl; —(CH₂)ₚCOR⁸; —(CH₂)ₚR⁸; F;
R⁵=lower alkyl; aralkyl; substituted aralkyl;
R⁶=lower alkyl; aralkyl; substituted aralkyl; —(CH₂)ₘCOR⁸; —(CH₂)_{q}R⁸;
R⁷=lower alkyl; aralkyl; substituted aralkyl; —(CH₂)ₘCOR⁸;
R⁸=lower alkyl; aralkyl; substituted aralkyl; aryl; substituted aryl; heteroaryl; substituted heteroaryl; hydroxy; lower alkoxy;
R⁹ is represented by any structure of the following formulae:

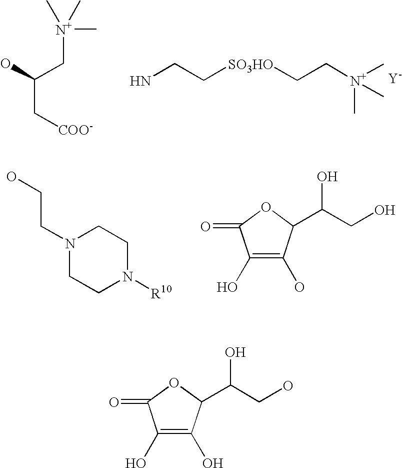

R¹⁰=hydrogen; lower alkyl; aralkyl or substituted aralkyl; aryl or substituted aryl;
Y⁻ represents the anion of a pharmaceutically acceptable acid;
n=0, 1; m=0, 1, 2; p=1, 2, 3; q=2, 3, 4; and
r=0, 1.

In another embodiment, the glutathione peroxidase mimetic or its isomer, metabolite, and/or salt thereof is represented by the compound of formula (III):

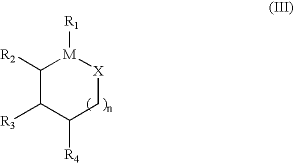

wherein,
   X is O or NH
   M is Se or Te
   n is 0-2
   R₁ is oxygen; and forms an oxo complex with M; or
R₁ is oxygen or NH; and
forms together with the metal, a 4-7 member ring, which optionally is substituted by an oxo or amino group; or
forms together with the metal, a first 4-7 member ring, which is optionally substituted by an oxo or amino group, wherein said first ring is fused with a second 4-7 member ring, wherein said second 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and
R₂, R₃ and R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R₄ together with the organometallic ring to which two of the substituents are attached, form a fused 4-7 member ring system wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; wherein R₄ is not an alkyl; and
wherein if R₂, R₃ and R₄ are hydrogen and R₁ forms an oxo complex with M, n is 0 then M is Te; or
if R₂, R₃ and R₄ are hydrogen and R₁ is an oxygen that forms together with the metal an unsubstituted, saturated, 5 member ring, n is 0 then M is Te; or
if R₁ is an oxo group, and n is 0, R₂ and R₃ form together with the organometallic ring a fused benzene ring, R₄ is hydrogen, then M is Se; or
if R₄ is an oxo group, and R₂ and R₃ form together with the organometallic ring a fused benzene ring, R₁ is oxygen, n is 0 and forms together with the metal a first 5 membered ring, substituted by an oxo group a to R₁, and said ring is fused to a second benzene ring, then M is Te;

In another embodiment, the glutathione peroxidase mimetic or its isomer, metabolite, and/or salt thereof is represented by the compound of formula (IV):

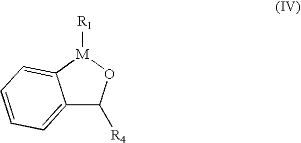

wherein, M, R₁ and R₄ are as described above.

In another embodiment, the glutathione peroxidase mimetic or its isomer, metabolite, and/or salt thereof is represented by the compound of formula (V):

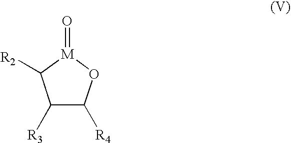

wherein, M, R₂, R₃ and R₄ are as described above;

In another embodiment, the glutathione peroxidase mimetic or its isomer, metabolite, and/or salt thereof is represented by the compound of formula (VI):

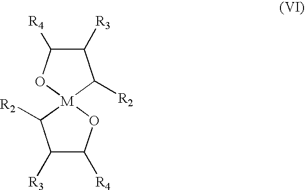

wherein, M, R₂, R₃ and R₄ are as described above;

In another embodiment, the glutathione peroxidase mimetic or its isomer, metabolite, and/or salt thereof is represented by the compound of formula (VII):

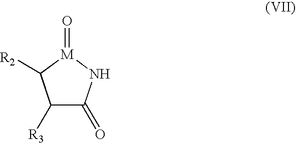

wherein, M, R₂, and R₃ are as described above.

In another embodiment, the glutathione peroxidase mimetic or its isomer, metabolite, and/or salt thereof is represented by the compound of formula (VIII):

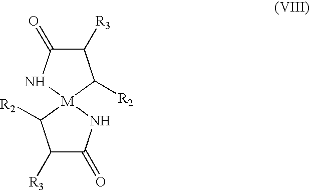

wherein, M, R₂, and R₃ are as described above.

In another embodiment, the glutathione peroxidase mimetic or its isomer, metabolite, and/or salt thereof is represented by the compound of formula (IX)

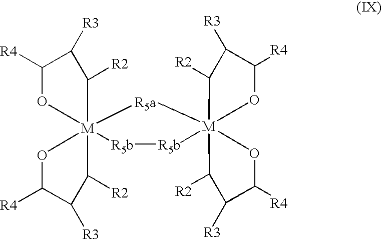

wherein,
M is Se or Te;
R₂, R₃ or R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R₄ together with the organometallic ring to which two of the substituents are attached, is a fused 4-7 member ring system, wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and
R₅ₐ or R_{5b} is one or more oxygen, carbon, or nitrogen atoms and forms a neutral complex with the chalcogen.

In another embodiment, the glutathione peroxidase mimetic or its isomer, metabolite, and/or salt thereof is represented by the compound of formula (X):

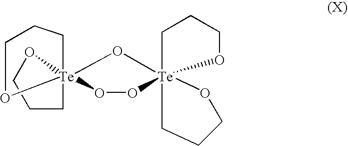

or their combination.

In another embodiment, the glutathione peroxidase mimetic or its isomer, metabolite, and/or salt thereof is represented by the compound of formula (XI):

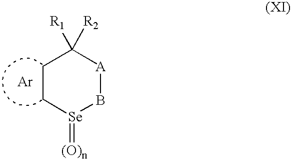

in which: R₁hydrogen; lower alkyl; optionally substituted aryl; optionally substituted lower aralkyl; R₂=hydrogen; lower alkyl; optionally substituted aryl; optionally substituted lower aralkyl; A=CO; (CR₃R₄)ₘ; B═NR₅; O; S; Ar=optionally substituted phenyl or an optionally substituted radical of formula:

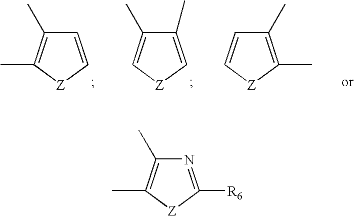

in which: Z=O; S; NR₅; R₃=hydrogen; lower alkyl; optionally substituted aryl; optionally substituted lower aralkyl R₄=hydrogen; lower alkyl; optionally substituted aryl; optionally substituted lower aralkyl; R₅=hydrogen; lower alkyl; optionally substituted aryl; optionally substituted lower aralkyl; optionally substituted heteroaryl; optionally substituted lower heteroaralkyl; CO(lower alkyl); CO(aryl); SO₂ (lower alkyl); SO₂(aryl); R₆=hydrogen; lower alkyl; optionally substituted aryl; optionally substituted lower aralkyl; optionally substituted heteroaryl; optionally substituted lower heteroaralkyl; trifluoromethyl;

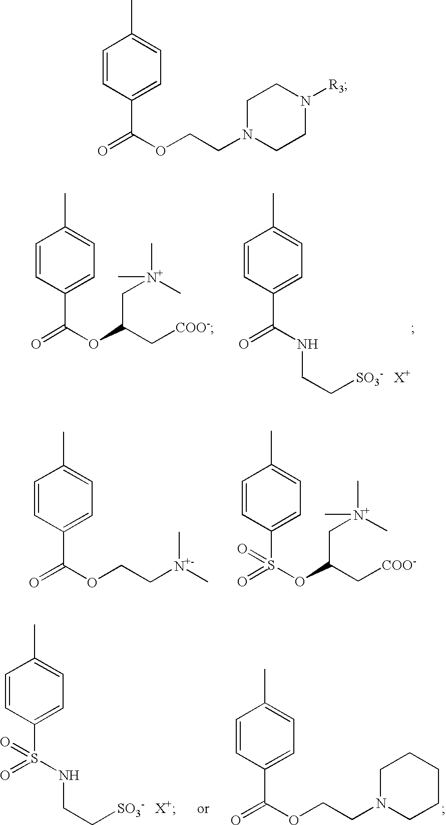

m=0 or 1; n=0 or 1; X⁺ represents the cation of a pharmaceutically acceptable base; and their pharmaceutically acceptable salts of acids or bases.

In other embodiments compounds useful for the purposes herein include 4,4-dimethyl-thieno-[3,2-e]-isoselenazine, 4,4-dimethyl-thieno-[3,2-e]-isoselenazine-1-oxide, 4,4-dimethyl-thieno-[2,3-e]-isoselenazine, and 4,4-dimethyl-thieno-[2,3-e]-isoselenazine-1-oxide.

In another embodiment, the glutathione peroxidase mimetic or its isomer, metabolite, and/or salt thereof is represented by the compound of formula (XII):

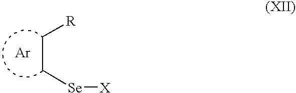

in which: R=hydrogen; —C(R₁R₂)-A-B;
R₁=lower alkyl; optionally substituted aryl; optionally substituted lower aralkyl;
R₂=lower alkyl: optionally substituted aryl: optionally substituted lower aralkyl;
A=CO; (CR₃R₄)ₙ;
B represents NR₅R₆; N⁺R₅R₆R₇Y⁻; OR₅; SR₅;
Ar=an optionally substituted phenyl group or an optionally substituted radical of

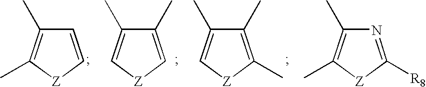

in which Z represents O; S; NR₅; when R═—C(R₁R₂)-A-B or Ar=a radical of formula

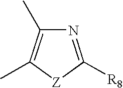

in which Z=O; S; NR₅; when R is hydrogen; X=Ar(R)—Se—; —S-glutathione; —S—N-acetylcysteine; —S-cysteine; —S-penicillamine; —S-albumin; —S-glucose;

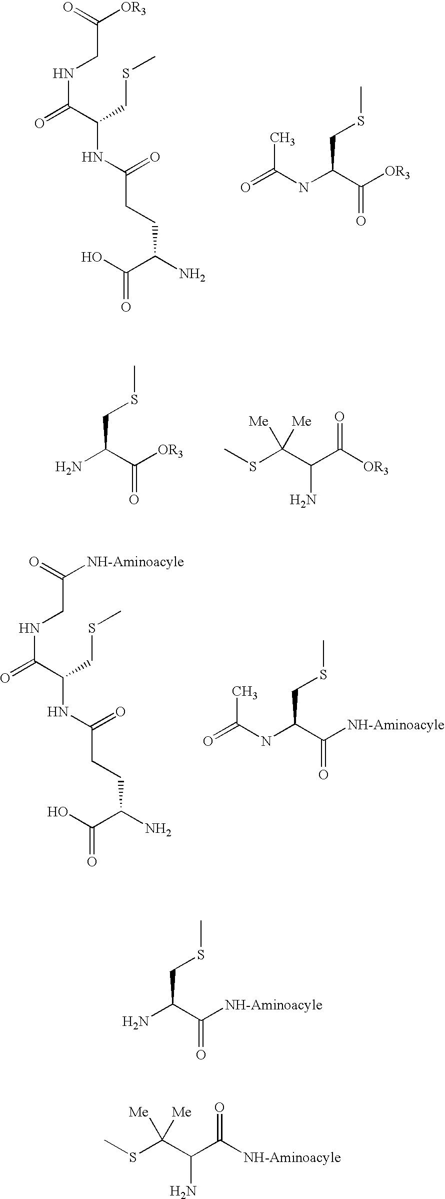

R₃=hydrogen; lower alkyl; optionally substituted aryl, optionally substituted lower aralkyl;
R₄=hydrogen; lower alkyl; optionally substituted aryl: optionally substituted lower aralkyl;
R₅=hydrogen; lower alkyl; optionally substituted aryl: optionally substituted lower aralkyl; optionally substituted heteroaryl; optionally substituted lower heteroaralkyl; CO(lower alkyl); CO(aryl); SO₂(lower alkyl); SO₂ (aryl);
R₆=hydrogen; lower alkyl; optionally substituted aryl; optionally substituted lower aralkyl; optionally substituted heteroaryl; optionally substituted lower heteroaralkyl;
R₇=hydrogen; lower alkyl; optionally substituted aryl: optionally substituted lower aralkyl; optionally substituted heteroaryl; optionally substituted lower heteroaralkyl;
R₈=hydrogen; lower alkyl; optionally substituted aryl; optionally substituted lower aralkyl; optionally substituted heteroaryl; optionally substituted lower heteroaralkyl; trifluoromethyl;

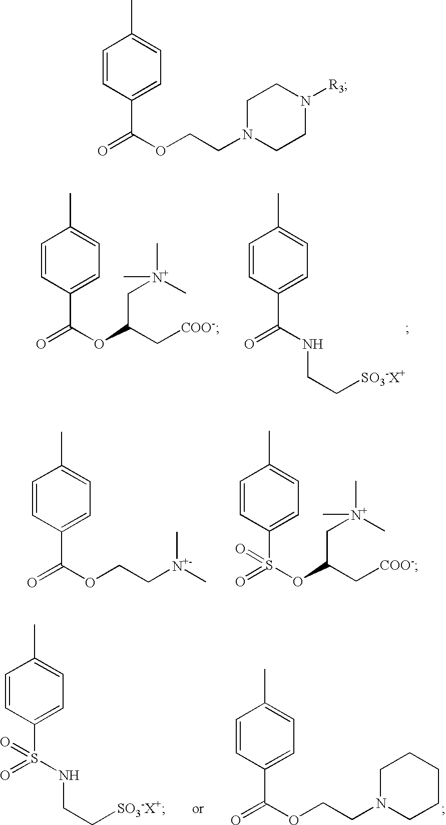

n=0 or 1; X⁺ represents the cation of a pharmaceutically acceptable base;
Y⁻ represents the anion of a pharmaceutically acceptable acid;
and their salts of pharmaceutically acceptable acids or bases.

In one embodiment, provided herein is a method of treating a dermatological condition in a subject, comprising the step of contacting the subject with glutathione peroxidase mimetic or its isomer, metabolite, and/or salt thereof and one or more of an age spots removing agents, keratoses removing agents, analgesics, anesthetics, antiacne agents, antibacterial agents, antiyeast agents, antifungal agents, antiviral agents, antiburn agents, antidandruff agents, antidermatitis agents, antipruritic agents antiperspirants, antiinflammatory agents, antihyperkeratolytic agents, antidryskin agents, antipsoriatic agents, antiseborrheic agents, antioxidants, vitamin C, astringents, softeners, emollient agents, coal tar, bath oils, sulfur, rinse conditioners, foot care agents, hair growth agents, powder, shampoos, skin bleaches, skin protectants, soaps, cleansers, antiaging agents, sunscreen agents, wart removers, vitamins, tanning agents, topical antihistamines, hormones, vasodilators, retinoids or combination thereof. In one embodiment, provided herein is a method of treating a dermatological condition in a subject, comprising the step of contacting the subject with glutathione peroxidase mimetic or its isomer, metabolite, and ascorbic acid, thereby increasing the endogenous antioxidant capability of the skin and scavanging free radicals from the skin.

In yet another embodiment, a method is provided for the treatment of psoriasis by administration to a subject a glutathione peroxidase mimetic or its isomer, metabolite, and/or salt thereof. In further embodiments, the administration is by a topical route. In other embodiments, any of the compounds of formulas (I) to (XII) above are useful for the treatment of psoriasis by administration to a subject. In other embodiments, administration is by a topical route. In another embodiment, a method is provided for the treatment of psoriasis by administering to a subject the compound of formula (I) above.

In other embodiments, pharmaceutical compositions for the treatment of the aforementioned dermatoses are provided comprising a compound of any one of formula (I) to (XII) in a pharmaceutically acceptable carrier or diluent. In some embodiments, the carrier or diluent is suitable for topical administration. In other embodiments, the carrier or diluent comprises liposomes. In further embodiments, the liposomes are formed from a hydroalcoholic medium. In a further embodiment, the pharmaceutical composition or liposome formulation comprises a compound of formula (I) above.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description

### DETAILED DESCRIPTION OF THE INVENTION

This invention relates in one embodiment to compositions and methods for the treatment of dermatological conditions with glutathione peroxidase and its mimetics.

In one embodiment, the term “dermatoses” refers to any skin defect or lesion on the skin. In one embodiment, the terms “dermatological condition” and “dermatoses” are interchangeable. In another embodiment, the dermatological conditions is scar formation. In another embodiment, the dermatological conditions is erythema. In another embodiment, the dermatological conditions is edema. In another embodiment, the dermatological conditions is pain. In another embodiment, the dermatological conditions is pruritus. In another embodiment, the dermatological conditions is eczema. In another embodiment, the dermatological conditions is erythroderma. In another embodiment, dermatoses referres to mycosis fungoides. In another embodiment, the dermatological conditions is pyoderma, gangrenosum. In another embodiment, the dermatological conditions is erythema multiforme. In one embodiment, dermatoses referres to rosacea. In another embodiment, the dermatological conditions is onychomycosis. In another embodiment, the dermatological conditions is acne. In another embodiment, the dermatological conditions is actinic keratosis. In another embodiment, the dermatological conditions is lentigines. In another embodiment, dermatoses referres to seborrheic keratosis. In another embodiment, the dermatological conditions is psoriasis. In another embodiment, the dermatological conditions is contact dermatitis. In another embodiment, the dermatological conditions is atopic dermatisis. In one embodiment dermatoses referres to a combination thereof.

In one embodiment, because molecular oxygen is virtually everywhere and it freely accepts electrons, oxygen-centered radicals are the most common mediators of cellular free radical reactions. Free oxygen-containing radicals are produced in another embodiment as a result of aerobic metabolism. In one embodiment, a very significant amount is generated as a result of photochemical reactions. Any organic or inorganic compound will absorb some UV radiation, and the absorbed energy will promote chemical reactions. In one embodiment, light causes the generation of oxygen-centered radicals. In another embodiment, the interaction of sunlight with organic or inorganic substrates on exposed skin result in one or more reactive oxygen species being produced on the skin. In another embodiment, the presence of oxygen radicals on the skin is responsible for a number of the undesirable effects of prolonged exposure to the sun. In one embodiment, the aging phenomenon observed in certain embodiments throughout the body is frequently observed prematurely on the skin as a result of photoaging, which accelerates the process of deterioration of elastin and collagen, among other effects. UV exposure causes in one embodiment, an increased risk of skin cancer of all types.

Oxidative stress refers in one embodiment to a loss of redox homeostasis (imbalance) with an excess of reactive oxidative species (ROS) by the singular process of oxidation. Both redox and oxidative stress are associated in another embodiment, with an impairment of antioxidant defensive capacity as well as an overproduction of ROS. In another embodiment, the methods and compositions of the invention are used in the treatment of complications or pathologies resulting from oxidative stress in subjects.

In another embodiment, injury to adult skin induces the release of various chemotactic factors that attract neutrophils and macrophages into the wound. The influx of these cells is beneficial, in one embodiment, since they play an important role in the defense against contaminating bacteria by phagocytosis, and in another embodiment, by the production and release of various proteinases and reactive oxygen species (ROS). This increased production of ROS is referred to in one embodiment as the “respiratory burst”.

In one embodiment, activated neutrophils and tissue macrophages use an NADPH cytochrome b-dependent oxidase for the reduction of molecular oxygen to superoxide anions. In another embodiment, fibroblasts, are also be stimulated to produce ROS in response to pro-inflammatory cytokines. In another embodiment, prolonged production of high levels of ROS cause severe tissue damage. In one embodiment, high levels of ROS cause DNA mutations that can lead to neoplastic transformation. Therefore and in one embodiment, cells in injured tissues must be able to protect themselves against the toxic effects of ROS. In one embodiment ROS-detoxifying enzymes have an important role in cutaneous wound repair. In another embodiment, the glutathione peroxidase mimetics provided in the compositions and compounds provided herein, replace the ROS detoxifying enzymes described herein.

In one embodiment, overproduction of reactive oxygen species (ROS) including hydrogen peroxide (H₂O₂), superoxide anion (O.₂⁻); nitric oxide (NO.) and singlet oxygen (¹O₂) creates an oxidative stress, resulting in the amplification of the inflammatory response. Self-propagating lipid peroxidation (LPO) against membrane lipids begins and endothelial dysfunction ensues. Endogenous free radical scavenging enzymes (FRSEs) such as superoxide dismutase (SOD), glutathione peroxidase (GPX) and catalase are, involved in the disposal of O.₂⁻ and H₂O₂. First, SOD catalyses the dismutation of O.₂⁻ to H₂O₂ and molecular oxygen (O₂), resulting in selective O.₂⁻ scavenging. Then, GPX and catalase independently decompose H₂O₂ to H₂O. In another embodiment, ROS is released from the active neutrophils in the inflammatory tissue, attacking DNA and/or membrane lipids and causing chemical damage, including in one embodiment, to healthy tissue. When in another embodiment, free radicals are generated in excess or when FRSEs are defective, H₂O₂ is reduced into hydroxyl radical (OH.), which is one of the highly reactive ROS responsible in one embodiment for initiation of lipid peroxidation of cellular membranes. In another embodiment, organic peroxide-induced lipid peroxidation is implicated as one of the essential mechanisms of toxicity in keratinocytes. In one embodiment, benzoyl peroxide, a topical agent, shows the ability to induce an inflammatory reaction mediated by oxidative stress in addition to its antibacterial activity, thereby increasing lipid peroxidation. In one embodiment, an indicator of the oxidative stress in the cell is the level of lipid peroxidation and its final product is MDA. In another embodiment the level of lipid peroxidation increases in inflammatory diseases. In one embodiment, the compounds provided herein and in another embodiment, are represented by the compounds of formula I-XII, are effective antioxidants, capable of reducing lipid peroxidation, or in another embodiment, are effective as anti-inflammatory agents.

In one embodiment, the effectiveness of the compounds provided herein derive from special structural features of the heterocyclic compounds provided herein. In one embodiment, having a large number of electrons in the π orbital overlap around the transition metal incorporated allows the formation of π-bonds and the donation of an electron to terminate free radicals formed by ROS. In one embodiment, the glutathione peroxidase mimetic used in the method of inhibiting or suppressing free radical formation, causing in another embodiment, lipid peroxidation and inflammation, is the product of formula (I):

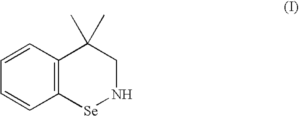

where nitrogen has 4 electrons in the p-orbital, thereby making 2 electrons available for π bonds; and each carbon has 2 electron in the p-orbital thereby making 1 electron available for π bonds; and selenium has 6 electrons in the p-orbital, thereby making 3 electrons available for π bonds, for a total of 7 electrons, since in another embodiment, the adjacent benzene ring removes two carbons from participating in the π-bond surrounding the metal. Upon a loss of electron by the transition metal, following termination of free radicals, the number of electrons in the π-bond overlap, is reduced to 6 π electron, a very stable aromatic sextet.

In one embodiment, a strong overexpression of heme oxigenase (HO) is observed in the skin of patients suffering from the inflammatory skin disease psoriasis. In another embodiment, inflammatory cells and keratinocytes of the hyperthickened epidermis are the major producers of HO. in psoriatic skin. In one embodiment, reactive oxygen species (ROS), are the inducers of HO-1 expression in inflamed skin.

In one embodiment, the strong expression of ROS-detoxifying enzymes in keratinocytes of normal skin or in cutaneous wounds in another embodiment, explains the observed resistance of keratinocytes to high doses of hydrogen peroxide.

In one embodiment, “rosacea” refers to a chronic and progressive cutaneous vascular disorder, primarily involving the malar and nasal areas of the face. Rosacea is characterized in another embodiment by flushing, erythema, papules, pustules, telanglectasia, facial edema, ocular lesions, and, in its most advanced and severe form, hyperplasia of tissue and sebaceous glands leading to rhinophyma. In one embodiment, the term “rhinophyma” refers to a florid overgrowth of the tip of the nose with hypervascularity and modularity and is an unusual progression of rosacea of unknown cause. In another embodiment, arachidonic acid is oxidized via the cyclooxygenase or lipoxygenase pathways, thereby, prostaglandins, leukotrines, and hydroxyeicosatetraenoic acid (HETE) are produced, which in one embodiment cause erythema, edema, and free radical production. In one embodiment, increasing the endogenous anti-oxidant activity of the skin, or in another embodiment, eliminating free radicals capable of contributing to lipid oxidation, using the compositions and methods provided herein, is effective in the treatment of rosacea and its subsequent pathologies.

In one embodiment, the dematologic condition is an allergic and inflammatory skin diseases. In another embodiment the dermatological condition is atopic dermatitis. In another embodiment, the dermatological conditions is urticaria and psoriasis, which are mediated by oxidative stress. In one embodiment, monocytes from patients with atopic dermatitis are primed to generate ROS in response to zymosan, a Toll-like receptor 2 (TLR2) ligand, indicating that Staphylococcus aureus may damage lesional skin of the disease by production of ROS. Mast cells generate mainly intracellular ROS following the aggregation of FceRI; these ROS act in one embodiment as secondary messengers in the induction of several biological responses. In another embodiment, the term “psoriasis” or “psoriasis vulgaris” refers to a chronic inflammatory skin disease characterized in another embodiment, by well-demarcated erythema and scaly plaques. In one embodiment, increased reactive oxygen species (ROS) and insufficient antioxidant activity have been determined in psoriatic lesions. In one embodiment, increasing the endogenous anti-oxidant activity of the skin, or in another embodiment, eliminating free radicals capable of contributing to the ROS/antioxidant imbalance, using the compositions and methods provided herein, is effective in the treatment of rosacea and its subsequent pathologies.

In another embodiment, compounds described herein block TNF-alpha activated expression of cell adhesion molecules, I-CAM and V-CAM, which may be essential for cellular migration. In another embodiment, such activity is effective in the treatment of psoriasis and other dermatoses in which TNF signaling mediated pathology of the diseases, based on inhibition of the induction of cell adhesion molecules and consequent migration of pro-inflammatory cells into inflamed tissue. Such activity is achieved in one embodiment by the topical application of compounds of formulas (I)-(XII) to the skin.

In one embodiment, the term “skin” refers to the epidermal layer of the skin and, in some cases, the dermal layer of the skin. In one embodiment, the epidermal layer of the skin is the outer (epithelial) layer and the stratum corneum, and the deeper connective tissue layer of the skin is called the dermis.

In one embodiment, exposure of mammalian skin to solar UV radiation, induces erythema, edema, sunburn cell formation, hyperplastic responses, photoaging and skin cancer development. In another embodiment, UV exposure, particularly UVB (290-320 nm), causes the generation of free radicals and related reactive oxygen species (ROS). In another embodiment, ROS generated as a consequence of UV exposure produces oxidative stress in the skin when formation exceeds the antioxidant defense ability of the target cell. Although the skin possesses an elaborate antioxidant defense system to deal with UV-induced oxidative stress, excessive exposure to UV can overwhelm in another embodiment, the cutaneous antioxidant capacity, leading to oxidative damage. Accordingly and in one embodiment, increasing the endogenic anti-oxidant ability of the skin, or applying to affected areas, using the compositions and methods provided herein, that are capable of scavenging the free radicals causing erythema, edema, sunburn cell formation, hyperplastic responses, photoaging, is effective in treating these pathologies.

In another embodiment, glutathione peroxidase, is important in the skin's defense mechanism against oxygen burst. In vitro and in vivo studies with the compound of formula I, show in one embodiment, that glutathione peroxidase is capable of protecting cells against reactive oxygen species.

Four types of GPx have been identified: cellular GPx (cGPx), gastrointestinal GPx, extracellular GPx, and phospholipid hydroperoxide GPx. cGPx, also termed in one embodiment, GPX1, is ubiquitously distributed. It reduces hydrogen peroxide as well as a wide range of organic peroxides derived from unsaturated fatty acids, nucleic acids, and other important biomolecules. At peroxide concentrations encountered under physiological conditions and in another embodiment, it is more active than catalase (which has a higher Kₘ for hydrogen peroxide) and is active against organic peroxides in another embodiment. Thus, cGPx represents a major cellular defense against toxic oxidant species.

Peroxides, including hydrogen peroxide (H₂O₂), are one of the main reactive oxygen species (ROS) leading to oxidative stress. H₂O₂ is continuously generated by several enzymes (including superoxide dismutase, glucose oxidase, and monoamine oxidase) and must be degraded to prevent oxidative damage. The cytotoxic effect of H₂O₂ is thought to be caused by hydroxyl radicals generated from iron-catalyzed reactions, causing subsequent damage to DNA, proteins, and membrane lipids.

In one embodiment, administration of GPx, a mimic thereof or its pharmaceutically acceptable salt, its functional derivative, its synthetic analog or a combination thereof, is used in the methods and compositions of the invention.

In one embodiment, the glutathione peroxidase mimetic is represented by formula I

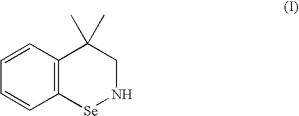

In one embodiment, the compound of formula (II), refers to benzisoselen-azoline or -azine derivatives represented by the following general formula:

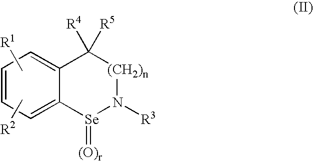

where: R¹ and R² are independently hydrogen; lower alkyl; OR⁶; —(CH₂)ₘNR⁶R⁷; —(CH₂)_{q}NH₂; —(CH₂)ₘNHSO₂ (CH₂)₂ NH₂; —NO₂; —CN; —SO₃H; —N⁺(R⁵)₂O⁻; F; Cl; Br; I; —(CH₂)ₘR⁸; —(CH₂)ₘCOR⁸; —S(O)NR⁶R⁷; —SO₂ NR⁶R⁷; —CO(CH₂)ₚCOR⁸; R⁹; R³=hydrogen; lower alkyl; aralkyl; substituted aralkyl; —(CH₂)ₘCOR⁸; —(CH₂)_{q}R⁸; —CO(CH₂)ₚCOR⁸; —(CH₂)ₘSO₂R⁸; —(CH₂)ₘS(O)R⁸; R⁴=lower alkyl; aralkyl; substituted aralkyl; —(CH₂)ₚCOR⁸; —(CH₂)ₚR⁸; F; R⁵=lower alkyl; aralkyl; substituted aralkyl; R⁶=lower alkyl; aralkyl; substituted aralkyl; —(CH₂)ₘCOR⁸; —(CH₂)_{q}R⁸; R⁷=lower alkyl; aralkyl; substituted aralkyl; —(CH₂)ₘCOR⁸; R⁸=lower alkyl; aralkyl; substituted aralkyl; aryl; substituted aryl; heteroaryl; substituted heteroaryl; hydroxy; lower alkoxy; R⁹; R⁹=

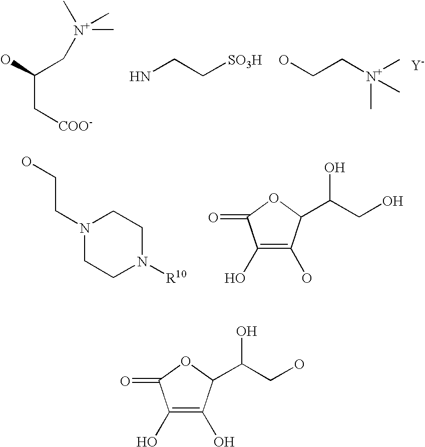

R¹⁰=hydrogen; lower alkyl; aralkyl or substituted aralkyl; aryl or substituted aryl;. Y⁻ represents the anion of a pharmaceutically acceptable acid; n=0, 1; m=0, 1, 2; p=1, 2, 3; q=2, 3, 4 and r=0, 1.

In one embodiment, “Alkyl” refers to monovalent alkyl groups preferably having from 1 to about 12 carbon atoms, more preferably 1 to 8 carbon atoms and still more preferably 1 to 6 carbon atoms. This term is exemplified by groups such as methyl, ethyl, n-propyl, isopropyl, n-butyl, isobutyl, tert-butyl, n-hexyl, n-octyl, tert-octyl and the like. The term “lower alkyl” refers to alkyl groups having 1 to 6 carbon atoms.

In another embodiment, “Aralkyl” refers to -alkylene-aryl groups preferably having from 1 to 10 carbon atoms in the alkylene moiety and from 6 to 14 carbon atoms in the aryl moiety. Such alkaryl groups are exemplified by benzyl, phenethyl, and the like.

“Aryl” refers in another embodiment, to an unsaturated aromatic carbocyclic group of from 6 to 14 carbon atoms having a single ring (e.g., phenyl) or multiple condensed rings (e.g., naphthyl or anthryl). Preferred aryls include phenyl, naphthyl and the like. Unless otherwise constrained by the definition for the individual substituent, such aryl groups can optionally be substituted with from 1 to 3 substituents selected from the group consisting of alkyl, substituted alkyl, alkoxy, alkenyl, alkynyl, amino, aminoacyl, aminocarbonyl, alkoxycarbonyl, aryl, carboxyl, cyano, halo, hydroxy, nitro, trihalomethyl and the like.

In one embodiment, the glutathione peroxidase or its isomer, metabolite, and/or salt thereof, used in the methods and compositions provided herein is an organoselenium compound. The term “organoselenium” refers in one embodiment to organic compound comprising at least one selenium atom. Preferred classes of organoselenium glutathione peroxidase mimetics include benzisoselenazolones, diaryl diselenides and diaryl selenides. In one embodiment, provided herein are compositions and methods of treating dermatoses associated pathologies with organoselenium compounds, thereby increasing endogenous anti-oxidant ability of the skin, or in another embodiment, scavenging free radicals causing the dermatoses associated pathologies.

In one embodiment compounds capable of scavanging free oxygen radicals, or in another embodiment, compounds capable of increasing endogenous anti-oxidant abilities are helpful in the treatment of dermatoses.

Accordingly and in one embodiment, provided herein is a composition for treating a dermatoses associated pathology in a subject, comprising: glutathione peroxidase or its isomer, metabolite, and/or salt thereof and pharmaceutically acceptable carrier or diluent.

In another embodiment, the glutathione peroxidase or its isomer, metabolite, and/or salt thereof used in the compositions and methods provided herein, is represented by the compound of formula III:

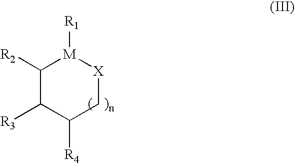

wherein,
the compound of formula 1 is a ring; and
   X is O or NH
   M is Se or Te
   n is 0-2
   R₁ is oxygen; and forms an oxo complex with M; or
   R₁ is oxygen or NH; and
forms together with the metal, a 4-7 member ring, which optionally is substituted by an oxo group; or
forms together with the metal, a first 4-7 member ring, which is optionally substituted by an oxo group, wherein said first ring is fused with a second 4-7 member ring, wherein said second 4-7 member ring is optionally substituted by alkyl, alkoxy, nitro, aryl, cyano, amino, halogen, or —NH(C═O)R or —SO₂R where R is alkyl or aryl;
R₂, R₃ and R₄ are independently hydrogen, alkyl, oxo, amino or together with the organometalic ring to which two of the substituents are attached, a fused 4-7 member ring system wherein said 4-7 member ring is optionally substituted by alkyl, alkoxy, nitro, aryl, cyano, amino, halogen, or —NH(C═O)R or —SO₂R where R is alkyl or aryl.
   In certain embodiments wherein R₄ is not an alkyl; and wherein if R₂, R₃ and R₄ are hydrogen and R₁ forms an oxo complex with M, n is 0 then M is Te; or if R₂, R₃ and R₄ are hydrogen and R₁ is an oxygen that forms together with the metal an unsubstituted, saturated, 5 member ring, n is 0 then M is Te; or if R₁ is an oxo group, and n is 0, R₂ and R₃ form together with the organometalic ring a fused benzene ring, R₄ is hydrogen, then M is Se; or if R₄ is an oxo group, and R₂ and R₃ form together with the organometalic ring a fused benzene ring, R₁ is oxygen, n is 0 and forms together with the metal a first 5 member ring, substituted by an oxo group a to R₁, and said ring is fused to a second benzene ring, then M is Te.

In one embodiment, a 4-7 member ring group refers to a saturated cyclic ring. In another embodiment the 4-7 member ring group refers to an unsaturated cyclic ring. In another embodiment the 4-7 member ring group refers to a heterocyclic unsaturated cyclic ring. In another embodiment the 4-7 member ring group refers to a heterocyclic saturated cyclic ring. In one embodiment the 4-7 member ring is unsubstituted. In one embodiment, the ring is substituted by one or more of the following: alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl.

In one embodiment, substituent groups may be attached via single or double bonds, as appropriate, as will be appreciated by one skilled in the art.

According to embodiments herein, the term alkyl as used throughout the specification and claims may include both “unsubstituted alkyls” and/or “substituted alkyls”, the latter of which may refer to alkyl moieties having substituents replacing hydrogen on one or more carbons of the hydrocarbon backbone. In another embodiment, such substituents may include, for example, a halogen, a hydroxyl, an alkoxyl, a silyloxy, a carbonyl, and ester, a phosphoryl, an amine, an amide, an imine, a thiol, a thioether, a thioester, a sulfonyl, an amino, a nitro, or an organometallic moiety. It will be understood by those skilled in the art that the moieties substituted on the hydrocarbon chain may themselves be substituted, if appropriate. For instance, the substituents of a substituted alkyl may include substituted and unsubstituted forms of amines, imines, amides, phosphoryls (including phosphonates and phosphines), sulfonyls (including sulfates and sulfonates), and silyl groups, as well as ethers, thioethers, selenoethers, carbonyls (including ketones, aldehydes, carboxylates, and esters), —CF₃, and —CN. Of course other substituents may be applied. In another embodiment, cycloalkyls may be further substituted with alkyls, alkenyls, alkoxys, thioalkyls, aminoalkyls, carbonyl-substituted alkyls, CF₃, and CN. Of course other substituents may be applied.

In one embodiment, the compound of formula III, used in the compositions and methods provided herein, is represented by any one of the following compounds or their combinations:

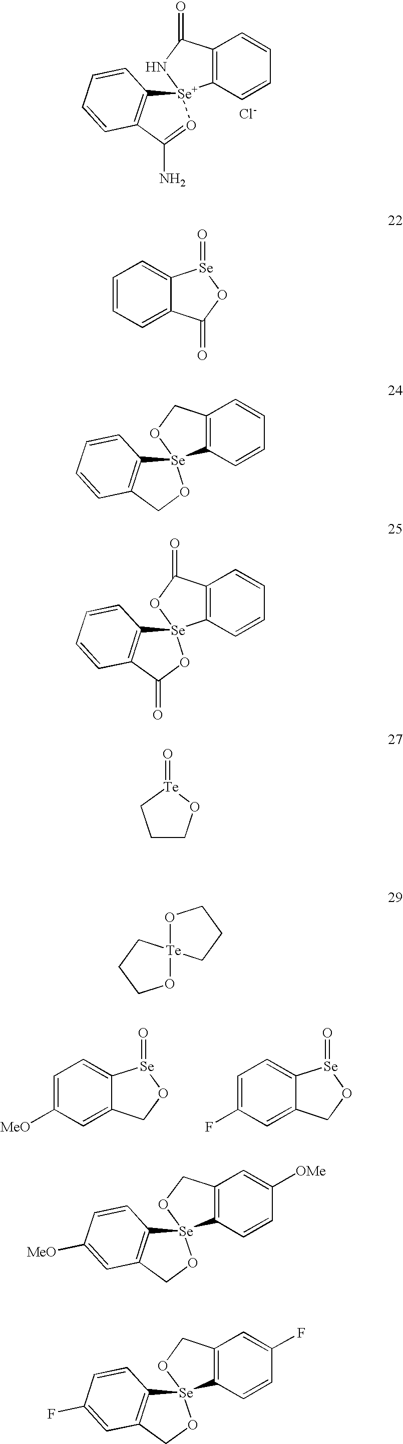

In another embodiment, a compound of formula IV is provided, wherein M, R₁ and R₄ are as described above.

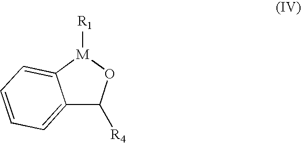

In another embodiment, a compound of formula V is provided, wherein M, R₂, R₃ and R₄ are as described above.

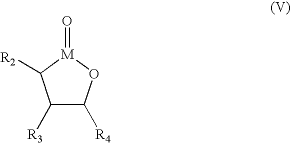

In another embodiment, a compound of formula VI is provided, wherein M, R₂, R₃ and R₄ are as described above.

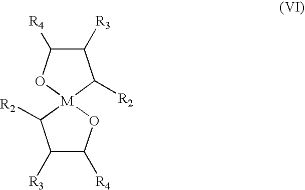

In another embodiment, a compound of formula (VII) is provided, wherein M, R₂ and R₃ are as described above.

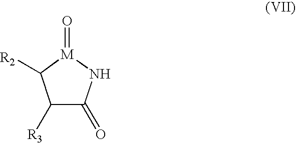

In another embodiment, a compound of formula VIII is provided, wherein M, R₂ and R₃ are as described above.

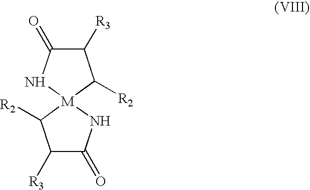

In another embodiment, the glutathione peroxidase or its isomer, metabolite, and/or salt thereof used in the compositions and methods provided herein, is represented by the compound of formula IX:

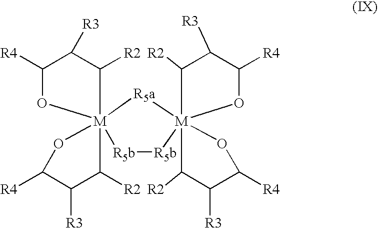

wherein,
M is Se or Te;

R₂, R₃ or R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R₄ together with the organometallic ring to which two of the substituents are attached, is a fused 4-7 membered ring system, wherein said 4-7 membered ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and

R₅ₐ or R_{5b} is one or more oxygen, carbon, or nitrogen atoms and forms a neutral complex with the chalcogen.

In one embodiment, the compound represented formula (IX), is represented by the compound of formula X:

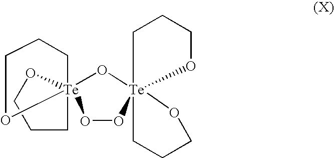

In another embodiment, the glutathione peroxidase mimetic or its isomer, metabolite, and/or salt thereof is represented by the compound of formula (XI):

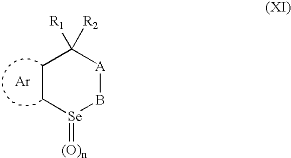

in which: R₁=hydrogen; lower alkyl; optionally substituted aryl; optionally substituted lower aralkyl; R₂=hydrogen; lower alkyl; optionally substituted aryl; optionally substituted lower aralkyl; A=CO; (CR₃R₄)ₘ; B═NR₅; O; S; Ar-optionally substituted phenyl or an optionally substituted radical of formula:

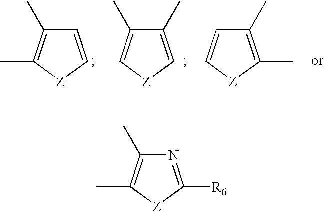

in which: Z=O; S; NR₅; R₃=hydrogen; lower alkyl; optionally substituted aryl; optionally substituted lower aralkyl R₄=hydrogen; lower alkyl; optionally substituted aryl; optionally substituted lower aralkyl; R₅=hydrogen; lower alkyl; optionally substituted aryl; optionally substituted lower aralkyl; optionally substituted heteroaryl; optionally substituted lower heteroaralkyl; CO(lower alkyl); CO(aryl); SO₂ (lower alkyl); SO₂ (aryl); R₆=hydrogen; lower alkyl; optionally substituted aryl; optionally substituted lower aralkyl; optionally substituted heteroaryl; optionally substituted lower heteroaralkyl; trifluoromethyl;

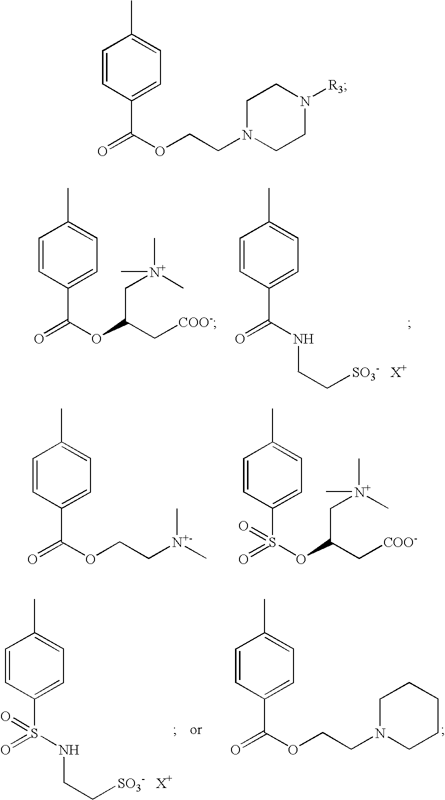

m=0 or 1; n=0 or 1; X⁺ represents the cation of a pharmaceutically acceptable base; and their pharmaceutically acceptable salts of acids or bases. In some embodiments, when B=NR₅ with R₅ is hydrogen, lower alkyl, optionally substituted lower aralkyl, CO(lower alkyl), and A=CO or (—CH₂—)ₘ, then Ar is different from an optionally substituted phenyl.

In other embodiments compounds useful for the purposes herein include 4,4-dimethyl-thieno-[3,2-e]-isoselenazine, 4,4-dimethyl-thieno-[3,2-e]-isoselenazine-1-oxide, 4,4-dimethyl-thieno-[2,3-e]-isoselenazine, and 4,4-dimethyl-thieno-[2,3-e]-isoselenazine-1-oxide.

In another embodiment, the glutathione peroxidase mimetic or its isomer, metabolite, and/or salt thereof is represented by the compound of formula (XII):

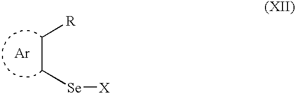

in which: R=hydrogen; —C(R₁R₂)-A-B;
R₁=lower alkyl; optionally substituted aryl; optionally substituted lower aralkyl;
R₂=lower alkyl: optionally substituted aryl: optionally substituted lower aralkyl;
A=CO; (CR₃R₄)ₙ;
B represents NR₅R₆; N⁺R₅R₆R₇Y⁻; OR₅; SR₅;
Ar=an optionally substituted phenyl group or an optionally substituted radical of

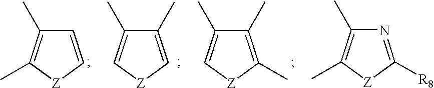

in which Z represents O; S; NR₅; when R═—C(R₁R₂)-A-B or Ar=a radical of formula

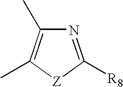

in which Z=O; S; NR₅; when R is hydrogen; X═Ar(R)—Se—; —S-glutathione; —S—N-acetylcysteine; —S-cysteine; —S-penicillamine; —S-albumin; —S-glucose;

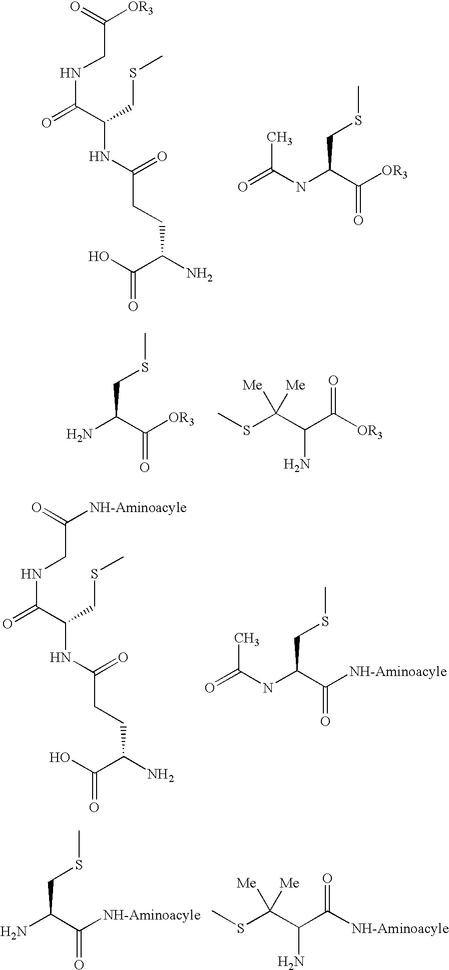

R₃=hydrogen; lower alkyl; optionally substituted aryl, optionally substituted lower aralkyl;
R₄=hydrogen; lower alkyl; optionally substituted aryl: optionally substituted lower aralkyl;
R₅=hydrogen; lower alkyl; optionally substituted aryl: optionally substituted lower aralkyl; optionally substituted heteroaryl; optionally substituted lower heteroaralkyl; CO (lower alkyl); CO (aryl); SO₂ (lower alkyl); SO₂ (aryl);
R₆=hydrogen; lower alkyl; optionally substituted aryl; optionally substituted lower aralkyl; optionally substituted heteroaryl; optionally substituted lower heteroaralkyl;
R₇=hydrogen; lower alkyl; optionally substituted aryl: optionally substituted lower aralkyl; optionally substituted heteroaryl; optionally substituted lower heteroaralkyl;
R₈=hydrogen; lower alkyl; optionally substituted aryl; optionally substituted lower aralkyl; optionally substituted heteroaryl; optionally substituted lower heteroaralkyl; trifluoromethyl;

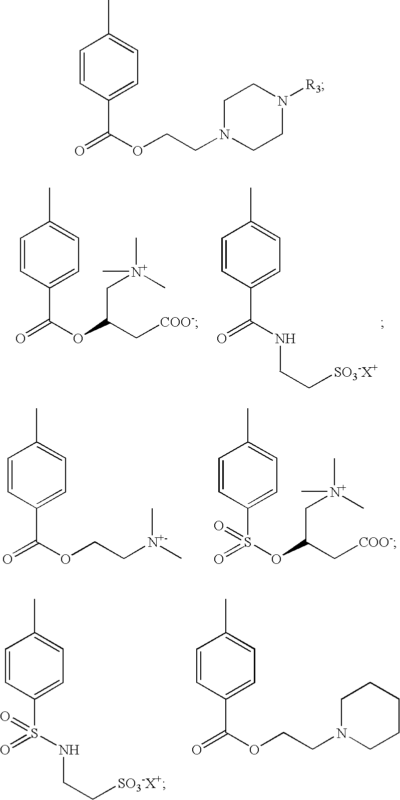

n=0 or 1; X⁺ represents the cation of a pharmaceutically acceptable base;
Y⁻ represents the anion of a pharmaceutically acceptable acid;
and their salts of pharmaceutically acceptable acids or bases.

In other embodiments, organoselenium compounds of formula (XII), include di[2-[2′-(1′-amino-2′-methyl)propyl]phenyl]-diselenide; di[2-[2′-(1′-amino-2′-methyl)propyl]phenyl]-diselenide dihydrochloride; di[2-[2′-(1′-ammonium-2′-methyl)propyl]phenyl]-diselenide di-paratoluenesulphonate; di[2-[2′-(1′-amino-2′-methyl)propyl]-4-methoxy]phenyl-diselenide; di[2-[2′-(1′-methylamino-2′-methyl)propyl]phenyl]-diselenide; di[2-[2′-(1′-methylamino-2′-methyl)propyl]phenyl]-diselenide dihydrochloride; di[2-[2′-(1′-dimethylamino-2′-methyl)propyl]phenyl]-diselenide; di[2-[2′-(1′-trimethylammonium-2′-methyl)propyl]phenyl]-diselenide di-paratoluenesulphonate; S-(N-acetyl-L-cysteinyl)-[2-[2′-(1′-amino-2′-methyl)-propyl]phenyl]-selenide; and S-glutathionyl-[2-[2′-(1′-amino-2′-methyl)-propyl]-phenyl]-selenide.

In one embodiment, the compounds represented by formula I-XII, mimic the in-vivo activity of glutathione peroxidase. The term “mimic” refers, in one embodiment to comparable, identical, or superior activity, in the context of conversion, timing, stability or overall performance of the compound, or any combination thereof.

Biologically active derivatives or analogs of the proteins described herein include in one embodiment peptide mimetics. These mimetics can be based, for example, on the protein's specific amino acid sequence and maintain the relative position in space of the corresponding amino acid sequence. These peptide mimetics possess biological activity similar to the biological activity of the corresponding peptide compound, but possess a “biological advantage” over the corresponding amino acid sequence with respect to, in one embodiment, the following properties: solubility, stability and susceptibility to hydrolysis and proteolysis.

Methods for preparing peptide mimetics include modifying the N-terminal amino group, the C-terminal carboxyl group, and/or changing one or more of the amino linkages in the peptide to a non-amino linkage. Two or more such modifications can be coupled in one peptide mimetic molecule. Other forms of the proteins and polypeptides described herein and encompassed by the claimed invention, include in another embodiment, those which are “functionally equivalent.” In one embodiment, this term, refers to any nucleic acid sequence and its encoded amino acid which mimics the biological activity of the protein, or polypeptide or functional domains thereof in other embodiments.

In one embodiment, the composition further comprises a carrier, excipient, lubricant, flow aid, processing aid or diluent, wherein said carrier, excipient, lubricant, flow aid, processing aid or diluent is a gum, starch, a sugar, a cellulosic material, an acrylate, calcium carbonate, magnesium oxide, talc, lactose monohydrate, magnesium stearate, colloidal silicone dioxide or mixtures thereof.

In another embodiment, the composition further comprises a binder, a disintegrant, a buffer, a protease inhibitor, a surfactant, a solubilizing agent, a plasticizer, an emulsifier, a stabilizing agent, a viscosity increasing agent, a sweetener, a film forming agent, or any combination thereof.

In one embodiment, the compositions provided herein are used for the treatment of dermatological conditions and may be present in the form of suspension or dispersion form in solvents or fats, in the form of a nonionic vesicle dispersion or else in the form of an emulsion, preferably an oil-in-water emulsion, such as a cream or milk, or in the form of an ointment, gel, cream gel, sun oil, solid stick, powder, aerosol, foam or spray. In another embodiment a liposome composition is provided.

In one embodiment, the composition is a particulate composition coated with a polymer (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, or intracranially. In another embodiment the administration is topical.

a tablet, a capsule, a solution, a suspension, a dispersion, an emulsion, an elixir, a gel, an ointment, a cream, or a suppository.

In another embodiment, the composition is in a form suitable for oral, intravenous, intraaorterial, intramuscular, subcutaneous, parenteral, transmucosal, transdermal, or topical administration. In one embodiment the composition is a controlled release composition. In another embodiment, the composition is an immediate release composition. In one embodiment, the composition is a liquid dosage form. In another embodiment, the composition is a solid dosage form.

In another embodiment, the compositions provided herein and methods of use therefor are suitable for oral, intraoral, rectal, parenteral, topical, epicutaneous, transdermal, subcutaneous, intramuscular, intranasal, sublingual, buccal, intradural, intraocular, intrarespiratory, nasal inhalation or a combination thereof. In one embodiment, the step of administering the compositions provided herein, in the methods provided herein is carried out as oral administration, or in another embodiment, the administration of the compositions provided herein is intraoral, or in another embodiment, the administration of the compositions provided herein is rectal, or in another embodiment, the administration of the compositions provided herein is parenteral, or in another embodiment, the administration of the compositions provided herein is topical, or in another embodiment, the administration of the compositions provided herein is epicutaneous, or in another embodiment, the administration of the compositions provided herein is transdermal, or in another embodiment, the administration of the compositions provided herein is subcutaneous, or in another embodiment, the administration of the compositions provided herein is intramuscular, or in another embodiment, the administration of the compositions provided herein is intranasal, or in another embodiment, the administration of the compositions provided herein is sublingual, or in another embodiment, the administration of the compositions provided herein is buccal, or in another embodiment, the administration of the compositions provided herein is intradural, or in another embodiment, the administration of the compositions provided herein is intraocular, or in another embodiment, the administration of the compositions provided herein is intrarespiratory, or in another embodiment, the administration of the compositions provided herein is nasal inhalation or in another embodiment, the administration of the compositions provided herein is a combination thereof.

In one embodiment, the method of the invention comprises administering a the compositions provided herein via an intradermal patch. The method in some embodiments also comprises administering the patch adjacent to the area of skin to be treated. As used herein a “patch” comprises at least the compositions provided herein and a covering layer, such that, the patch can be placed over the area of skin to be treated. In another embodiment, the patch is designed to maximize delivery of the compositions provided herein through the stratum corneum and into the epidermis or dermis, reduce lag time, promote uniform absorption, and reduce mechanical rub-off.

In some embodiments, the method comprises administering a topical formulation of the compositions provided herein to an affected site of skin. In some embodiments, topical administration according to the present invention comprises aerosol, cream, foam, gel, liquid, ointment, paste, powder, shampoo, spray, patch, disk, or dressing. Any of the foregoing topical formulations can in other embodiments comprise liposomes wherein the compound is entrapped in phospholipid vesicles.

The compounds utilized in the methods and compositions of the present invention may be present in the form of free bases in one embodiment or pharmaceutically acceptable acid addition salts thereof in another embodiment. In one embodiment, the term “pharmaceutically-acceptable salts” embraces salts commonly used to form alkali metal salts and to form addition salts of free acids or free bases. The nature of the salt is not critical, provided that it is pharmaceutically-acceptable. Where appropriate, suitable pharmaceutically-acceptable acid addition salts of compounds of Formula I-XII are prepared in another embodiment, from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, beta-hydroxybutyric, salicylic, galactaric and galacturonic acid. Suitable pharmaceutically-acceptable base addition salts include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, in another embodiment, the appropriate acid or base with the compound.

In one embodiment, the term “pharmaceutically acceptable carriers” includes, but is not limited to, may refer to 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In one embodiment the level of phosphate buffer used as a pharmaceutically acceptable carrier is between about 0.01 to about 0.1M, or between about 0.01 to about 0.09M in another embodiment, or between about 0.01 to about 0.08M in another embodiment, or between about 0.01 to about 0.07M in another embodiment, or between about 0.01 to about 0.06M in another embodiment, or between about 0.01 to about 0.05M in another embodiment, or between about 0.01 to about 0.04M in another embodiment, or between about 0.01 to about 0.03M in another embodiment, or between about 0.01 to about 0.02M in another embodiment, or between about 0.01 to about 0.015 in another embodiment.

In one embodiment, the compounds of this invention for the uses embodied herein may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds. Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations comprising the compositions used in one embodiment in the methods provided herein, can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition described in the embodiments provided herein, can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed, for example, with the free amino groups of the compounds embodied herein), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In one embodiment, the compositions described herein, which are used in another embodiment, in the methods provided herein, further comprise a carrier, an excipient, a lubricant, a flow aid, a processing aid or a diluent.

The active agent is administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

Alternatively, targeting therapies may be used in another embodiment, to deliver the active agent more specifically to certain types of cell, by the use of targeting systems such as antibodies or cell specific ligands. Targeting may be desirable in one embodiment, for a variety of reasons, e.g. if the agent is unacceptably toxic, or if it would otherwise require too high a dosage, or if it would not otherwise be able to enter the target cells.

The compositions of the present invention for the uses embodied herein are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Controlled or sustained release compositions for the uses embodied herein include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In one embodiment, the composition for the uses embodied herein can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

In one embodiment, liposomes (vesicles) are completely closed lipid bilayer membranes containing an entrapped aqueous volume. Liposomes in one embodiment may be unilamellar vesicles (possessing a single membrane bilayer) or multilamellar vesicles (onion-like structures characterized by multiple membrane bilayers, each separated from the next by an aqueous layer). Liposomes smaller than about 200 nm usually only consist of one bilayer (unilamellar liposomes) but larger liposomes can contain concentric layers of lipid or several smaller liposomes can be formed inside large liposomes. These larger multicompartmented liposomes are known as multilamellar liposomes. Multivesicular liposomes are liposomes containing multiple non-concentric chambers within each liposome particle, resembling a “foam-like” matrix.

The original liposome preparation of Bangham et al. (J. Mol. Biol., 1965, 12 pp. 238-252) involves suspending phospholipids in an organic solvent which is then evaporated to dryness leaving a phospholipid film on the reaction vessel. Next, an appropriate amount of aqueous phase is added, the mixture is allowed to “swell,” and the resulting liposomes which consist of multilamellar vesicles (MLVs) are dispersed by mechanical means. MLVs so formed may be used in the practice of the present invention.

Another class of multilamellar liposomes embodied herein are characterized as having substantially equal lamellar solute distribution. This class of liposomes is denominated as stable plurilamellar vesicles (SPLV) as defined in U.S. Pat. No. 4,522,803 to Lehk, et al., reverse phase evaporation vesicles (REV) as described in U.S. Pat. No. 4,235,871 to Papahadjopoulos et al., monophasic vesicles as described in U.S. Pat. No. 4,558,579 to Fountain, et al., and frozen and thawed multilamellar vesicles (FATMLV) wherein the vesicles are exposed to at least one freeze and thaw cycle; this procedure is described in Bally et al., PCT Publication No. 87/00043, Jan. 15, 1987, entitled “Multilamellar Liposomes Having Improved Trapping Efficiencies”; these references are incorporated herein by reference.

Liposomes are comprised of lipids; the term lipid as used herein shall mean any suitable material resulting in a bilayer such that a hydrophobic portion of the lipid material orients toward the interior of the bilayer while a hydrophilic portion orients toward the aqueous phase. Exemplary but non-limiting lipids which can be used in the liposome formulations of the present invention are the phospholipids such as phosphatidylcholine (PC) and phosphatidylglycerol (PG), more particularly dimyristoylphosphatidylcholine (DMPC) and dimyristoylphosphatidylglycerol (DMPG). Liposomes may be formed and vesiculated using DMPG, or DMPG mixed with DMPC in, for example, a 3:7 mole ratio, respectively.

During preparation of the liposomes, organic solvents may be used to suspend the lipids. Suitable organic solvents are those with intermediate polarities and dielectric properties, which solubilize the lipids, and include but are not limited to halogenated, aliphatic, cycloaliphatic, or aromatic-aliphatic hydrocarbons, such as benzene, chloroform, methylene chloride, or alcohols, such as methanol, ethanol, and solvent mixtures such as benzene:methanol (70:30). As a result, solutions (mixtures in which the lipids and other components are uniformly distributed throughout) containing the lipids are formed. Solvents are generally chosen on the basis of their biocompatability, low toxicity, and solubilization abilities.

In further embodiments of liposome formulations of the compounds and compositions embodied here for the purposes hereof, such liposomes include soft vesicles, that are formed in a hydroalcoholic medium containing C₂-C₄ alcohols. Such liposomes when applied to the skin, change their size by fusing together as a result of the change in solvent ratio. In the formulation, the vesicle size does not change since the ratio between the solvents is constant. Penetration and evaporation of the alcohol following application to the skin allows the transition from small to large vesicles, which grow in size until a film is formed. In such embodiments, liposomes comprise vesicles in a size range up to about 1 micrometer, but can range from nanometers to millimeters. Such compositions comprise from 0.5% to 10% phospholipids, from 20% to 50% of ethanol, from 0 to 20% propylene glycol, at least 20% water, and the glutathione peroxidase mimetic or combination thereof, where the combined ethanol and propylene glycol content does not exceed 70%. In one embodiment, the glutathione peroxidase mimetic is the compound of formula (I). In other embodiments, compounds of formulas (II)-(XII) are contained therein. In other embodiments, such liposomes comprise 22 to 70% of a combination of the ethanol and propylene glycol, and more than 20% water. In still other embodiments, the amount of ethanol is between 20 and 50 weight-% of the composition, the content of water being at least about 25 weight-%. In yet other embodiment, a short chain C₃-C₄ alcohol can be used in addition to ethanol, such as but not limited to propanol, isopropanol, butanol, iso-butanol and t-butanol. In non-limiting examples of such liposomes, the composition can comprise 0.5% to 10% phospholipids, from 5% to 35% of a C₃ or C₄ alcohol, 15 to 30% ethanol, wherein the combined content of ethanol and C₃ or C₄ alcohol is at least 20 wt. % and not more than 40 wt. %, up to 20 wt. % glycol, such as propylene glycol, at least 20% water, encapsulating one or a combination of glutathione peroxidase mimetics described herein. In one embodiment, the glutathione peroxidase mimetic is the compound of formula (I). In other embodiments, compounds of formulas (II)-(XII) are contained therein. In another embodiment, the liposomal composition comprises from 0.5% to 10% phospholipids, from 5% to 35% of a C₃ or C₄ alcohol, 15% to 30% ethanol, where the C₃ or C₄ alcohol is at least 20 wt. % and not more than 40 wt. %, up to 20 wt. % glycol, at least 20% water and glutathione peroxidase mimetic such as but not limited to the compound of formula (I). By means well know in the art, the foregoing compositions are made by vigorous mixing or stirring into a colloid system containing the liposomal vesicles.

The phospholipids of such liposomes can comprise one or more for the following non-limiting examples: phosphatidylcholine, hydrogenated phosphatidylcholine, phosphatidic acid, phosphatidylserine, phosphatidylethanolamine, phosphatidyglycerol or phosphatidylinositol. Typically soya phospholipids such as Phospholipon 90 (PL-90) is used. The concentration of phospholipid ranges between about 0.5-10% w/w. Cholesterol at concentrations ranging between about 0.1-1% can also be added to the preparation. Examples of alcohols which can be used are: ethanol and isopropyl alcohol. Examples of glycols are propylene glycol and Transcutol™. The source of the phospholipids can be egg, soybean, semi-synthetics, and synthetics. Non ionic surfactants can be combined with the phospholipids in these preparations e.g. PEG-alkyl ethers (Brij-52). Cationic lipids like cocoamides, POE alkyl amines, dodecylamine, cetrimide and like.

The concentration of alcohol (such as ethanol or a C₃ or C₄ alcohol) in the final product ranges from about 20-50%. The concentration of the non-aqueous phase (alcohol and glycol combination) may range between about 22 to 70%. The rest of the carrier contains water and possible additives. For preparation of a formulation for application of such liposomes to the skin, the liposomes can be incorporated in various carriers such as: PVP/VA (gels, membranes, solutions), PVP (gels, membranes, solutions) carbomer gels, polaxomer (gels, solutions), emulsions, creams, Pluronic F127 or Tetronic gels and the like, cellulose derivatives gels, PL-90ant extract gels (aloe vera gel etc), and the like.

In other embodiments, such compositions for the uses embodied herein are in one embodiment liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral.

In another embodiment, the compositions for the uses embodied herein comprise one or more, pharmaceutically acceptable carrier materials.

In one embodiment, the carriers for use within such compositions are biocompatible, and in another embodiment, biodegradable. In other embodiments, the formulation may provide a relatively constant level of release of one active component. In other embodiments, however, a more rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duration of release and the nature of the condition to be treated suppressed or inhibited.

In one embodiment, the compositions of the invention are administered in conjunction with one or more therapeutic agents. These agents are in other embodiments, age spots removing agents, keratoses removing agents, analgesics, anesthetics, antiacne agents, antibacterial agents, antiyeast agents, antifungal agents, antiviral agents, antiburn agents, antidandruff agents, antidermatitis agents, antipruritic agents antiperspirants, antiinflammatory agents, antihyperkeratolytic agents, antidryskin agents, antipsoriatic agents, antiseborrheic agents, astringents, softeners, emollient agents, coal tar, bath oils, sulfur, rinse conditioners, foot care agents, hair growth agents, powder, shampoos, skin bleaches, skin protectants, soaps, cleansers, antiaging agents, sunscreen agents, wart removers, vitamins, tanning agents, topical antihistamines, hormones, vasodilators and retinoids.

In one embodiment, the compositions provided herein, are used in the methods described herein. Accordingly and in another embodiment, provided herein is a method of treating a dermatoses associated pathology in a subject, comprising the step of administering to said subject a therapeutically effective amount of a composition comprising glutathione peroxidase or its isomer, metabolite, and/or salt thereof and pharmaceutically acceptable carrier or diluent, thereby removing reactive oxygen species.

In one embodiment, the term “administering” refers to bringing a subject in contact with the compositions provided herein. For example, in one embodiment, the compositions provided herein are suitable for oral administration, whereby bringing the subject in contact with the composition comprises ingesting the compositions. In another embodiment, the compositions provided herein are suitable for topical administration, whereby administering the composition using a patch in another embodiment brings the subject in contact. In one embodiment, the compositions provided herein are in a cream form and are applied directly to the affected area on the subject's skin, thereby bringing the subject in contact with the compositions provided herein. A person skilled in the art would readily recognize that the methods of bringing the subject in contact with the compositions provided herein, will depend on many variables such as, without any intention to limit the modes of administration; the skin condition treated, age, pre-existing conditions, other agents administered to the subject, the severity of symptoms, location of the affected are and the like. In one embodiment, provided herein are embodiments of methods for administering the compounds of the present invention to a subject, through any appropriate route, as will be appreciated by one skilled in the art.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

In one embodiment, the methods provided herein, using the compositions provided herein, further comprise contacting the subject with one or more additional agent. In another embodiment, the one or more additional agent is age spots removing agents, keratoses removing agents, analgesics, anesthetics, antiacne agents, antibacterial agents, antiyeast agents, antifungal agents, antiviral agents, antiburn agents, antidandruff agents, antidermatitis agents, antipruritic agents antiperspirants, antiinflammatory agents, antihyperkeratolytic agents, antidryskin agents, antipsoriatic agents, antiseborrheic agents, astringents, softeners, emollient agents, coal tar, bath oils, sulfur, rinse conditioners, foot care agents, hair growth agents, powder, shampoos, skin bleaches, skin protectants, soaps, cleansers, antiaging agents, sunscreen agents, wart removers, vitamins, tanning agents, topical antihistamines, hormones, vasodilators and retinoids.

Accordingly and in one embodiment, provided herein is a method of treating psoriasis in a subject, comprising the step of contacting the subject with glutathione peroxidase or its isomer, metabolite, and/or salt thereof and one or more of methotrexate; infliximab; etanercept, or adalimumab, thereby increasing endogenous antioxidant activity, removing free radicals and reducing inflammation on the skin.

In another embodiment, provided herein is a method of treating contact dermatitis in a subject, comprising contacting the subject with a topical cream, at the affected area, the cream comprising glutathione peroxidase or its isomer, metabolite, and/or salt thereof and one or more of Olopatadine hydrochloride: TAK-427 (2-[6-[[3-[4-(diphenylmethoxy)piperidino]propyl]amino]imidazo[1,2-b]pyridazin-2-yl]-2-methylpropionic acid dihydrate); itraconazole (ITZ); or a topical steroid, thereby increasing endogenous antioxidant activity, removing free radicals, reducing inflammation on the skin, and providing an antihistamine.

A person skilled in the art would readily recognize that combination therapy as described in the methods and compositions provided herein, could be administered either simultaneously or consecutively and so long as they are administered for the same condition, would be encompassed herein.

Having described preferred embodiments of the invention with reference to the accompanying drawings, it. is to be understood that the invention is not limited to the precise embodiments, and that various changes and modifications may be effected therein by those skilled in the art without departing from the scope or spirit of the invention as defined in the appended claims.

What is claimed is:

1. A composition for treating a dermatological condition in a subject, comprising: a glutathione peroxidase mimetic or its isomer, metabolite, and/or salt thereof and pharmaceutically acceptable carrier or diluent.

2. The composition of claim 1, wherein said glutathione peroxidase mimetic or its isomer, metabolite, and/or salt thereof is a compounds represented by formula I:

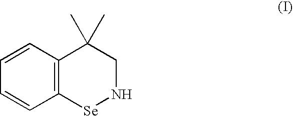

3. The composition of claim 1, wherein said glutathione peroxidase mimetic or its isomer, metabolite, and/or salt thereof is a benzisoselen-azoline or -azine derivative represented by the following general formula II: 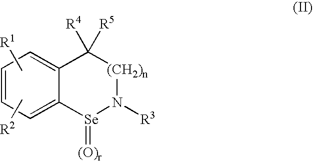
wherein R¹ and R² are independently hydrogen; lower alkyl; OR⁶; —(CH₂)ₘNR⁶R⁷; —(CH₂)_{q}NH₂; —(CH₂)ₘNHSO₂(CH₂)₂NH₂; —NO₂; —CN; —SO₃H; —N⁺(R⁵)₂O⁻; F; Cl; Br; I; —(CH₂)ₘR⁸; —(CH₂)ₘCOR⁸; —S(O)NR⁶R⁷; —SO₂ NR⁶R⁷; —CO(CH₂)ₚCOR⁸; R⁹;
R³=hydrogen; lower alkyl; aralkyl; substituted aralkyl; —(CH₂)ₘCOR⁸; —(CH₂)_{q}R⁸; —CO(CH₂)ₚCOR⁸; —(CH₂)ₘSO₂R⁸; —(CH₂)ₘS(O)R⁸;
R⁴=lower alkyl; aralkyl; substituted aralkyl; —(CH₂)ₚCOR⁸; —(CH₂)ₚR⁸; F;
R⁵=lower alkyl; aralkyl; substituted aralkyl;
R⁶=lower alkyl; aralkyl; substituted aralkyl; —(CH₂)ₘCOR⁸; —(CH₂)_{q}R⁸;
R⁷=lower alkyl; aralkyl; substituted aralkyl; —(CH₂)ₘCOR⁸;
R⁸=lower alkyl; aralkyl; substituted aralkyl; aryl; substituted aryl; heteroaryl; substituted heteroaryl; hydroxy; lower alkoxy;
R⁹ is represented by any structure of the following formulae: 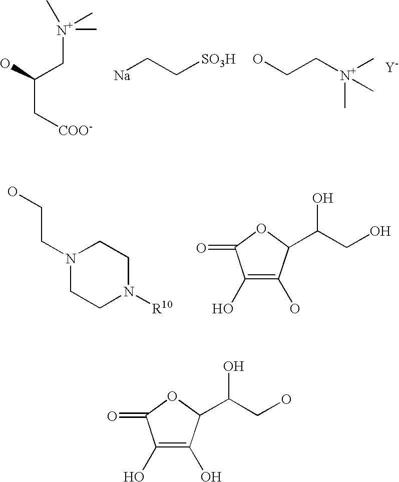
R¹⁰=hydrogen; lower alkyl; aralkyl or substituted aralkyl; aryl or substituted aryl;
Y represents the anion of a pharmaceutically acceptable acid;
n=0, 1; m=0, 1, 2; p=1, 2, 3; q=2, 3, 4; and
r=0, 1.

4. The composition of claim 1, wherein said glutathione peroxidase mimetic is a organoselenium compound, ebselen or their combination.

5. The composition of claim 1, wherein the dermatological condition is scar formation, erythema, edema, pain, pruritus, eczema, erythroderma, mycosis fungoides, pyoderma gangrenosum, erythema multiforme, rosacea, onychomycosis, acne, actinic keratosis, lentigines, seborrheic keratosis, psoriasis, contact dermatitis, atopic dermatisis or a combination thereof.

6. The composition of claim 1, wherein the dermatological condition is psoriasis.

7. The composition of claim 1, wherein the glutathione peroxidase mimetic or its isomer, metabolite, and/or salt thereof is the compound represented by formula (I).

8. The composition of claim 1, wherein the glutathione peroxidase mimetic or its isomer, metabolite, and/or salt thereof is represented by the compound of formula III: 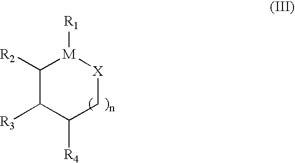
wherein,
the compound of formula 1 is a ring; and
X is O or NH
M is Se or Te
n is 0-2
R₁ is oxygen; and forms an oxo complex with M; or
R₁ is oxygen or NH; and
forms together with the metal, a 4-7 member ring, which optionally is substituted by an oxo or amino group; or
forms together with the metal, a first 4-7 member ring, which is optionally substituted by an oxo or amino group, wherein said first ring is fused with a second 4-7 member ring, wherein said second 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and
R₂, R₃ and R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R₄ together with the organometallic ring to which two of the substituents are attached, form a fused 4-7 member ring system wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, NR^{A}R^{B} or —SO₂R where R^{A} and R^{B} are independently H, alkyl or aryl; wherein R₄ is not an alkyl; and
wherein if R₂, R₃ and R₄ are hydrogen and R₁ forms an oxo complex with M, n is 0 then M is Te; or
if R₂, R₃ and R₄ are hydrogen and R₁ is an oxygen that forms together with the metal an unsubstituted, saturated, 5 member ring, n is 0 then M is Te; or
if R₁ is an oxo group, and n is 0, R₂ and R₃ form together with the organometallic ring a fused benzene ring, R₄ is hydrogen, then M is Se; or
if R₄ is an oxo group, and R₂ and R₃ form together with the organometallic ring a fused benzene ring, R₁ is oxygen, n is 0 and forms together with the metal a first 5 member ring, substituted by an oxo group (1 to R₁, and said ring is fused to a second benzene ring, then M is Te.

9. The composition of claim 8, wherein the compound of formula III is represented by the compounds:

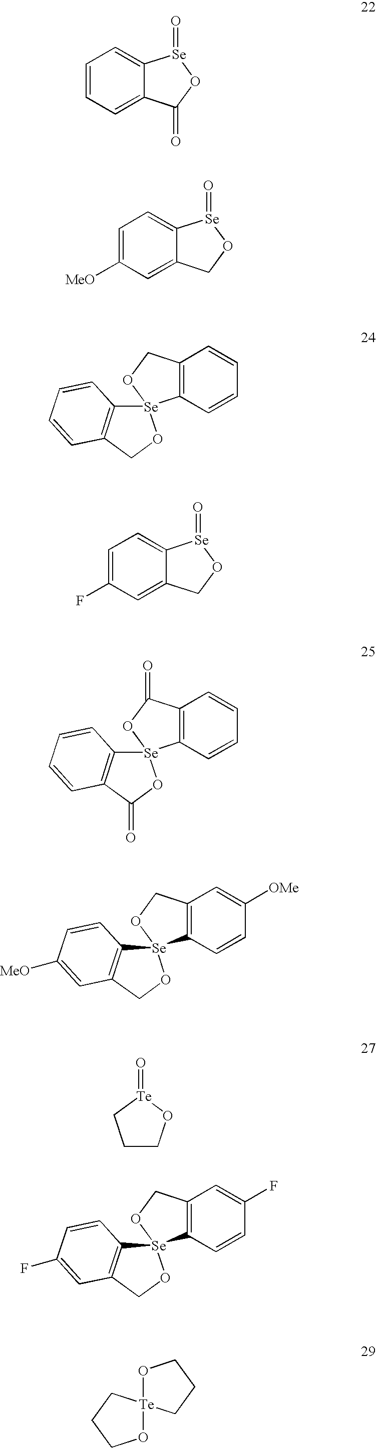

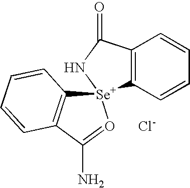

10. The composition of claim 1, wherein the glutathione peroxidase mimetic is represented by the compound of formula IV: 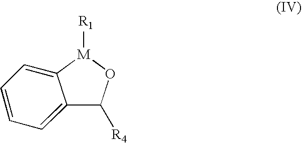
wherein, M, R₁ and R₄ are as described above.

11. The composition of claim 1, wherein the glutathione peroxidase mimetic is represented by the compound of formula V: 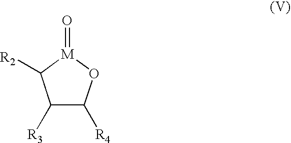
wherein, M, R₂, R₃ and R₄ are as described above.

12. The composition of claim 1, wherein the glutathione peroxidase mimetic is represented by the compound of formula VI: 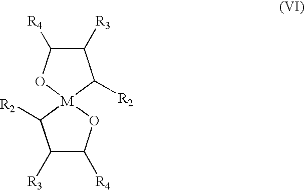
wherein, M, R₂, R₃ and R₄ are as described above.

13. The composition of claim 1, wherein the glutathione peroxidase mimetic is represented by the compound of formula VII: 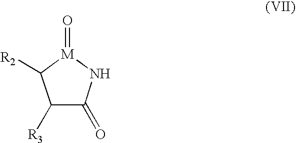
wherein, M, R₂, and R₃ are as described above.

14. The composition of claim 1, wherein the glutathione peroxidase mimetic is represented by the compound of formula VIII: 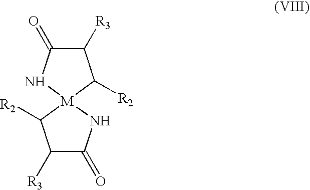
wherein, M, R₂, and R₃ are as described above.

15. The composition of claim 1, wherein the glutathione peroxidase mimetic or its isomer, metabolite, and/or salt thereof is represented by the compound of formula IX: 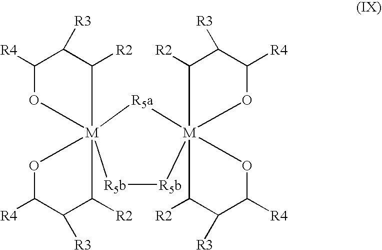
wherein,
M is Se or Te;
R₂, R₃ or R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R₄ together with the organometallic ring to which two of the substituents are attached, is a fused 4-7 member ring system, wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and
R₅ₐ or R_{5b} is one or more oxygen, carbon, or nitrogen atoms and forms a neutral complex with the chalcogen.

16. The composition of claim 1, wherein the glutathione peroxidase mimetic is represented by the compound of formula X:

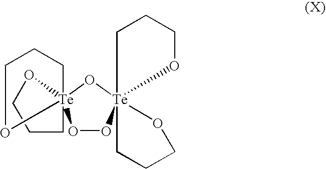

17. The composition of claim 1 wherein the glutathione peroxidase mimetic or its isomer, metabolite, and/or salt thereof is represented by the compound of formula (XI): 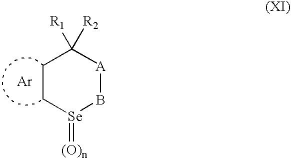
in which: R₁=hydrogen; lower alkyl; optionally substituted aryl; optionally substituted lower aralkyl; R₂=hydrogen; lower alkyl; optionally substituted aryl; optionally substituted lower aralkyl; A=CO; (CR₃R₄)ₘ; B=NR₅; O; S; Ar=optionally substituted phenyl or an optionally substituted radical of formula: 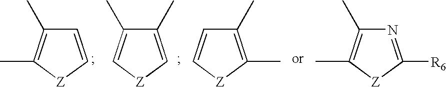
in which: Z=O; S; NR₅; R₃=hydrogen; lower alkyl; optionally substituted aryl; optionally substituted lower aralkyl R₄=hydrogen; lower alkyl; optionally substituted aryl; optionally substituted lower aralkyl; R₅=hydrogen; lower alkyl; optionally substituted aryl; optionally substituted lower aralkyl; optionally substituted heteroaryl; optionally substituted lower heteroaralkyl; CO(lower alkyl); CO(aryl); SO₂ (lower alkyl); SO₂ (aryl); R₆=hydrogen; lower alkyl; optionally substituted aryl; optionally substituted lower aralkyl; optionally substituted heteroaryl; optionally substituted lower heteroaralkyl; trifluoromethyl; 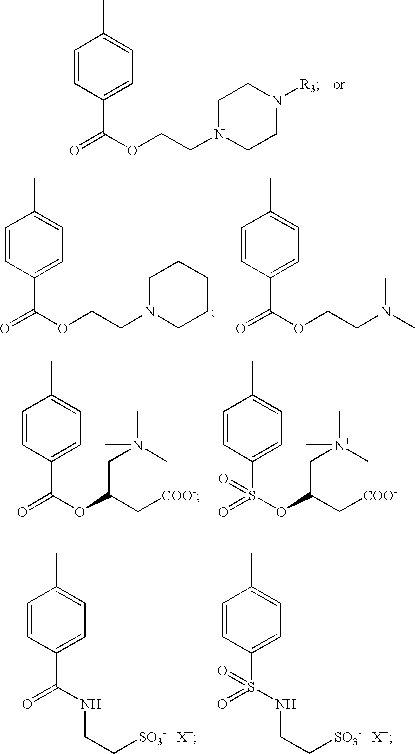
m=0 or 1; n=0 or 1; X⁺ represents the cation of a pharmaceutically acceptable base; and their pharmaceutically acceptable salts of acids or bases.

18. The composition of claim 17, wherein the compound comprises 4,4-dimethyl-thieno-[3,2-e]-isoselenazine, 4,4-dimethyl-thieno-[3,2-e]-isoselenazine-1-oxide, 4,4-dimethyl-thieno-[2,3-e]-isoselenazine, or 4,4-dimethyl-thieno-[2,3-e]-isoselenazine-1-oxide.

19. The composition of claim 1 wherein the glutathione peroxidase mimetic or its isomer, metabolite, and/or salt thereof is represented by the compound of formula (XII): 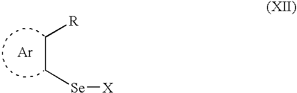
in which: R=hydrogen; —C(R₁R₂)-A-B;
R₁=lower alkyl; optionally substituted aryl; optionally substituted lower aralkyl;
R₂=lower alkyl: optionally substituted aryl: optionally substituted lower aralkyl;
A=CO; (CR₃R₄)ₙ;
B represents NR₅R₆; N⁺R₅R₆R₇Y⁻; OR₅; SR₅;
Ar=an optionally substituted phenyl group or an optionally substituted radical of 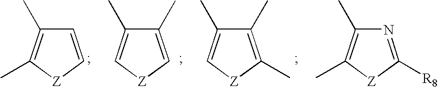
in which Z represents O; S; NR₅; when R=—C(R₁R₂)-A-B or Ar=a radical of formula 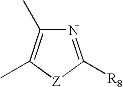
in which Z=O; S; NR₅; when R is hydrogen; X═Ar(R)—Se—; —S-glutathione; —S—N-acetylcysteine; —S-cysteine; —S-penicillamine; —S-albumin; —S-glucose; 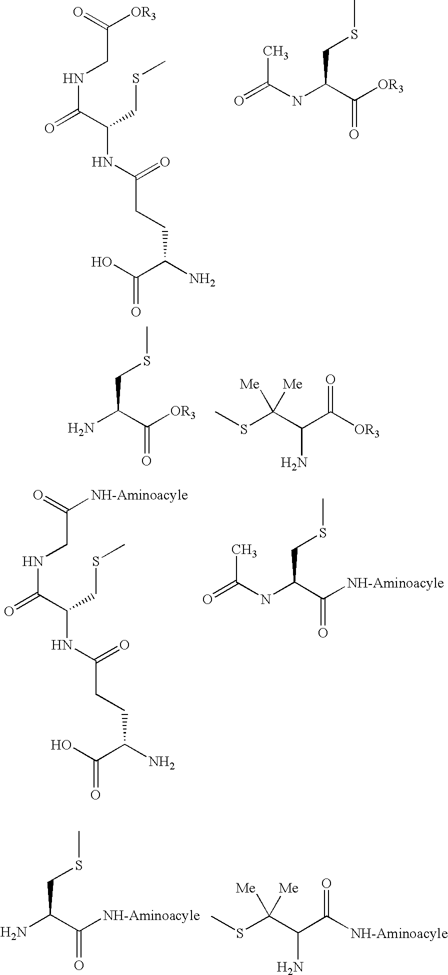
R₃=hydrogen; lower alkyl; optionally substituted aryl, optionally substituted lower aralkyl;
R₄=hydrogen; lower alkyl; optionally substituted aryl: optionally substituted lower aralkyl;
R₅hydrogen; lower alkyl; optionally substituted aryl: optionally substituted lower aralkyl; optionally substituted heteroaryl; optionally substituted lower heteroaralkyl; CO (lower alkyl); CO (aryl); SO₂ (lower alkyl); SO₂ (aryl);
R₆=hydrogen; lower alkyl; optionally substituted aryl; optionally substituted lower aralkyl;
optionally substituted heteroaryl; optionally substituted lower heteroaralkyl;
R₇=hydrogen; lower alkyl; optionally substituted aryl: optionally substituted lower aralkyl;
optionally substituted heteroaryl; optionally substituted lower heteroaralkyl;
R₈=hydrogen; lower alkyl; optionally substituted aryl; optionally substituted lower aralkyl;
optionally substituted heteroaryl; optionally substituted lower heteroaralkyl; trifluoromethyl; 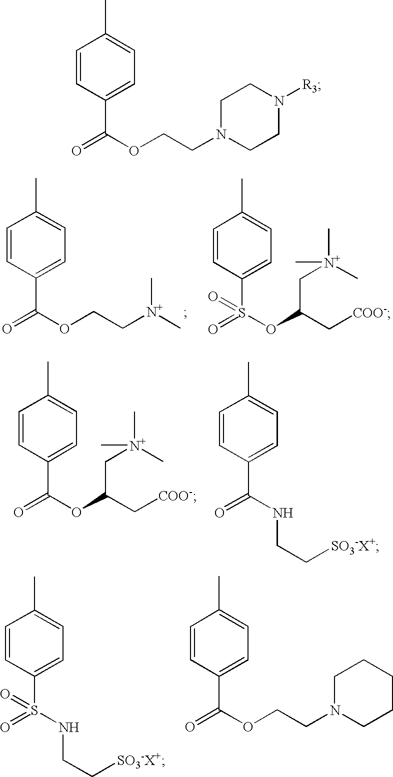
n=0 or 1; X⁺ represents the cation of a pharmaceutically acceptable base;
Y⁻ represents the anion of a pharmaceutically acceptable acid;
and their salts of pharmaceutically acceptable acids or bases.

20. The composition of claim 19 wherein the compound comprises di[2-[2′-(1′-amino-2′-methyl)propyl]phenyl]-diselenide; di[2-[2′-(1′-amino-2′-methyl)propyl]phenyl]-diselenide dihydrochloride; di[2-[2′-(1′-ammonium-2′-methyl)propyl]phenyl]-diselenide di-paratoluenesulphonate; di[2-[2′-(1′-amino-2′-methyl)propyl]-4-methoxy]phenyl-diselenide; di[2-[2′-(1′-methylamino-2′-methyl)propyl]phenyl]-diselenide; di[2-[2′-(1′-methylamino-2′-methyl)propyl]phenyl]-diselenide dihydrochloride; di[2-[2′-(1′-dimethylamino-2′-methyl)propyl]phenyl]-diselenide; di[2-[2′-(1′-trimethylammonium-2′-methyl)propyl]phenyl]-diselenide di-paratoluenesulphonate; S-(N-acetyl-L-cysteinyl)-[2-[2′-(1′-amino-2′-methyl)-propyl]phenyl]-selenide; or S-glutathionyl-[2-[2′-(1′-amino-2′-methyl)-propyl]-phenyl]-selenide.

21. The composition of claim 1, further comprising a carrier, an excipient, a lubricant, a flow aid, a processing aid or a diluent.

22. The composition of claim 21, wherein said carrier, excipient, lubricant, flow aid, processing aid or diluent is a gum, a starch, a sugar, a cellulosic material, an acrylate, calcium carbonate, magnesium oxide, talc, lactose monohydrate, magnesium stearate, colloidal silicone dioxide or mixtures thereof.

23. The composition of claim 1, comprising a binder, a disintegrant, a buffer, a protease inhibitor, a surfactant, a solubilizing agent, a plasticizer, an emulsifier, a stabilizing agent, a viscosity increasing agent, a sweetener, a film forming agent, or any combination thereof.

24. The composition of claim 1, wherein the composition is a controlled release composition.

25. The composition of claim 1, wherein the composition is an immediate release composition.

26. The composition of claim 1, comprising one or more additional agent for treating a dermatological in the subject.

27. The composition of claim 26, whereby the one or more additional agent is age spots removing agents, keratoses removing agents, analgesics, anesthetics, antiacne agents, antibacterial agents, antiyeast agents, antifungal agents, antiviral agents, antiburn agents, antidandruff agents, antidermatitis agents, antipruritic agents antiperspirants, antiinflammatory agents, antihyperkeratolytic agents, antidryskin agents, antipsoriatic agents, antiseborrheic agents, astringents, softeners, emollient agents, coal tar, bath oils, sulfur, rinse conditioners, foot care agents, hair growth agents, powder, shampoos, skin bleaches, skin protectants, soaps, cleansers, antiaging agents, sunscreen agents, wart removers, vitamins, tanning agents, topical antihistamines, hormones, vasodilators and retinoids.

28. A method of treating a dermatological condition in a subject, comprising the step of contacting said subject with a therapeutically effective amount of a composition comprising a glutathione peroxidase mimetic or its isomer, metabolite, and/or salt thereof and pharmaceutically acceptable carrier or diluent.

29. The method of claim 28, whereby the composition is any one of the compositions of claims 2-27.

30. The method of claim 28, whereby, said contacting comprises contacting via oral, intraoral, rectal, parenteral, topical, epicutaneous, transdermal, subcutaneous, intramuscular, intranasal, sublingual, buccal, intradural, intraocular, intrarespiratory, nasal inhalation or a combination thereof.

31. The method of claim 28, whereby the composition is a liquid formulated as a topical solution, spray, mist, foam or drops.

32. The method of claim 28 wherein the composition comprises liposomes containing the a glutathione peroxidase mimetic or its isomer, metabolite, and/or salt.

33. The method of claim 32 wherein the composition comprises a hydroalcoholic liposome.

34. A method for inhibiting or suppressing a dermatological condition in a subject, comprising the step of contacting said subject with a glutathione peroxidase mimetic or its isomer, metabolite, and/or salt thereof and pharmaceutically acceptable carrier or diluent, thereby removing reactive oxygen species.

35. A method for reducing the incidence of a dermatological condition in a subject, comprising the step of contacting said subject with glutathione peroxidase mimetic or its isomer, metabolite, and/or salt thereof and pharmaceutically acceptable carrier or diluent, thereby removing reactive oxygen species.

36. The method of claim 34 or 35, whereby the glutathione peroxidase mimetic or its isomer, metabolite, and/or salt thereof comprises the compound represented by any one of formulas I-XII, or any combination thereof.

37. The method of any one of claims 28-36, whereby the dermatological condition is scar formation, erythema, edema, pain, pruritus, eczema, erythroderma, mycosis fungoides, pyoderma gangrenosum, erythema multiforme, rosacea, onychomycosis, acne, actinic keratosis, lentigines, seborrheic keratosis, psoriasis, contact dermatitis, atopic dermatisis or a combination thereof.

38. The method of any one of claims 28-37, whereby, said contacting comprises contacting via oral, intraoral, rectal, parenteral, topical, epicutaneous, transdermal, subcutaneous, intramuscular, intranasal, sublingual, buccal, intradural, intraocular, intrarespiratory, nasal inhalation administration, or a combination thereof

39. The method of any one of claims 28-38, comprising contacting the subject with one or more additional agent, which is not glutathione peroxidase mimetic or its isomer, metabolite, and/or salt thereof.

40. The method of claim 39, whereby the one or more additional agent not a glutathione peroxidase mimetic or its isomer, metabolite, and/or salt thereof, is age spots removing agents, keratoses removing agents, analgesics, anesthetics, antiacne agents, antibacterial agents, antiyeast agents, antifungal agents, antiviral agents, antiburn agents, antidandruff agents, antidermatitis agents, antipruritic agents antiperspirants, antiinflammatory agents, antihyperkeratolytic agents, antidryskin agents, antipsoriatic agents, antiseborrheic agents, astringents, softeners, emollient agents, coal tar, bath oils, sulfur, rinse conditioners, foot care agents, hair growth agents, powder, shampoos, skin bleaches, skin protectants, soaps, cleansers, antiaging agents, sunscreen agents, wart removers, vitamins, tanning agents, topical antihistamines, hormones, vasodilators and retinoids.
